SEQUENCE LISTING (21 sequences)

SEQ ID NO: 1 - RNA, 39 nt, synthetic construct
  source 1..39: mol_type = other RNA
ggcatcagcctgcccatcaagcccagctgcctgcagagg

SEQ ID NO: 2 - protein, 13 aa, Mus musculus
GISLPIKPSCLQR

SEQ ID NO: 3 - RNA, 36 nt, synthetic construct
  source 1..36: mol_type = other RNA
ggcatcagcctgcccatcgccagcgccggcgacagg

SEQ ID NO: 4 - protein, 12 aa, Mus musculus
GISLPIASAGDR

SEQ ID NO: 5 - RNA, 39 nt, synthetic construct; identical to SEQ ID NO: 1
  source 1..39: mol_type = other RNA

SEQ ID NO: 6 - protein, 13 aa, Rattus rattus
GISLPIKPSCLQR

SEQ ID NO: 7 - RNA, 36 nt, synthetic construct; identical to SEQ ID NO: 3
  source 1..36: mol_type = other RNA

SEQ ID NO: 8 - protein, 12 aa, Rattus rattus
GISLPIASAGDR

SEQ ID NO: 9 - RNA, 42 nt, Canis lupus
  source 1..42: mol_type = mRNA
gacatctcgcttccgatcgagaagcccttctgcctccaggga

SEQ ID NO: 10 - protein, 14 aa, Canis lupus
DISLPIGKPFCLQG

SEQ ID NO: 11 - RNA, 39 nt, Canis lupus
  source 1..39: mol_type = mRNA
gacatctcgcttccgatcgcctcacctgcccgccgataa

SEQ ID NO: 12 - protein, 12 aa, Canis lupus
DISLPIASPARR

SEQ ID NO: 13 - RNA, 42 nt, Felis catus
  source 1..42: mol_type = mRNA
gacatctctctcctcattgagaagcccttctgtctacaagga

SEQ ID NO: 14 - protein, 14 aa, Felis catus
DISLLIEKPFCLQG

SEQ ID NO: 15 - RNA, 36 nt, Felis catus
  source 1..36: mol_type = mRNA
gacatctctctcctcattgcctcacctgcctgctga

SEQ ID NO: 16 - protein, 11 aa, Felis catus
DISLLIASPAC

SEQ ID NO: 17 - RNA, 42 nt, Homo sapiens
  source 1..42: mol_type = mRNA
gacttcgccctgcccgtggagaagcccctgtgcctgcagaga

SEQ ID NO: 18 - protein, 14 aa, Homo sapiens
DFALPVEKPLCLQR

SEQ ID NO: 19 - RNA, 36 nt, Homo sapiens
  source 1..36: mol_type = mRNA
gacttcgccctgcccgtggccagtctggcctgcaga

SEQ ID NO: 20 - protein, 12 aa, Homo sapiens
DFALPVASLACR

SEQ ID NO: 21 - protein, 7 aa, synthetic construct
RRRRRRG

(19) United States
(12) Patent Application Publication
(10) Pub. No.: US 2025/0064907 A1
(43) Pub. Date: Feb. 27, 2025

(54) THERAPEUTIC VACCINES FOR TREATMENT OF CHRONIC INFLAMMATORY DISEASES

(71) Applicant: ATHEROVAX, LLC, Lebanon, OR (US)

(72) Inventors: Patrick IVERSEN, Grand Junction, CO (US); Nodar KIPSHIDZE, New York, NY (US); Nickolas KIPSHIDZE, New York, NY (US)

(21) Appl. No.: 18/813,220

(22) Filed: Aug. 23, 2024

Related U.S. Application Data
(60) Provisional application No. 63/578,504, filed on Aug. 24, 2023.

(51) Int. Cl. A61K 39/00; A61P 1/04; A61P 37/02

(52) U.S. Cl. CPC A61K 39/0008; A61P 1/04; A61P 37/02; A61K 2039/53; A61K 2039/54

(57) ABSTRACT

Described herein are nucleic acid and peptide vaccines comprising sTNFR2 sequences complementary to variants of companion animal TNFR2 genes and methods for treating human or companion animal subjects for chronic inflammatory diseases.

### CROSS-REFERENCE TO RELATED APPLICATIONS

This application claims priority to U.S. Provisional Patent Application No. 63/578,504, filed on Aug. 24, 2023, which is incorporated by reference herein in its entirety.

This application is related to U.S. patent application Ser. No. 18/332,873, filed on Jun. 12, 2023, and published as U.S. Patent Application Publication No. US 2024/0050542 A1 on Feb. 15, 2024, and International Patent Application No. PCT/US2023/068270, filed on Jun. 12, 2023, and published as International Patent Application Publication No. WO 2023/244957 A1 on Dec. 21, 2023, each of which is incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “889990-0003-US02_sequence_listing_xml_16-AUG-2024.xml,” was created on Aug. 16, 2024, contains 21 sequences, has a file size of 20.0 kilobytes (20,480 bytes), and is incorporated by reference in its entirety into the specification.

### BACKGROUND

While acute inflammation is a normal and necessary response of the immune system to injury or infection, chronic inflammation is an ongoing and prolonged inflammatory response that can persist for weeks, months, or even years. Unlike acute inflammation, which helps the body heal, chronic inflammation is nearly always detrimental and is associated with a wide range of health conditions. Chronic inflammation contributes to the development and progression of cardiovascular diseases (e.g., atherosclerosis), diabetes, rheumatoid arthritis, inflammatory bowel diseases (e.g., Crohn's disease, ulcerative colitis), certain types of cancer, Alzheimer's disease, and chronic respiratory diseases. Chronic inflammation can affect brain function and alter neurotransmitter levels, contributing to mood disturbances associated with mental health disorders such as depression and anxiety. Chronic inflammation has been associated with accelerated aging processes and an increased risk of age-related diseases, such as age-related macular degeneration and sarcopenia.

Tumor necrosis factor alpha (TNFα) plays a significant role in chronic inflammation. TNFα is a cytokine, a type of signaling molecule produced by immune cells, and it plays a crucial role in regulating the body's immune response. While TNFα is an essential part of the immune system's defense against infections and injury, excessive or prolonged production of this cytokine can lead to chronic inflammation and contribute to the development and progression of various inflammatory diseases.

TNFα is observed extracellularly as a soluble (sTNFα) homotrimeric cytokine identified as a pro-inflammatory cytokine. The free, unbound, sTNFα is rapidly degraded with a half-life of 0.9 to 2.3 hours and is associated with acute inflammation. sTNFα bound to soluble TNF receptor II (STNFR2) is protected from degradation which serves to prolong the actions of sTNFα and the sTNFα: sTNFR2 complex can mediate chronic inflammation. This assertion is supported by the association of sTNFR2 with increased mortality and morbidity in many human diseases with chronic inflammatory disorders.

TNFα has become an important therapeutic target. Drugs that specifically block TNFα activity, known as TNF inhibitors (infliximab, adalimumab, golimumab, etanercept, and certolizumab), have been developed and used to treat certain inflammatory conditions. These medications can help reduce inflammation and alleviate symptoms in individuals with conditions like rheumatoid arthritis, psoriasis, and inflammatory bowel diseases but their efficacy can be limited by development of antidrug antibodies and neutralizing antibodies. In addition, the cost of these therapies exceeds $75,000 per year making them unattainable in low-income populations. At present, there are no therapeutic interventions targeting sTNFR2.

Studies evaluating expression of sTNFR2 in chronic inflammatory disease are limited because multiple forms of sTNFR2 can be produced in the human body. The membrane bound TNFR2 can become sTNFR2 by proteolytic cleavage by the TNFα converting enzyme (TACE) or from alternate exon use in processing of TNFR2 pre-mRNA in which exon 7 is excluded or in which exon 7 (Δ7 sTNFR2) and exon 7 and 8 (Δ7,8 sTNFR2) are excluded. The amino acids encoded by exon 7 are in the transmembrane domain so excluding these amino acids results in a soluble translated protein. All of these sTNFR2 variants can bind sTNFα. See Iversen et al., Front. Cardiovasc. Med. 10:1206541 (2023).

Current TNFα inhibitors including Infliximab (Remicade), adalimumab (Humira), Etanercept (Enbrel), and certolizumab (Cimzia) directly bind to TNFα resulting in immediate inactivation of free TNFα activity. These therapeutics are not recommended for individuals with chronic infections and can attenuate the beneficial effects of vaccination.

Vaccines are often large antigens containing numerous epitopes which can result in “immune confusion” because both immunodominant and subdominant epitopes are presented to the immune system. Large antigen vaccines presenting self-antigens carry significant liabilities for off-target activities resulting in adverse autoimmune activities.

What is needed is a sTNFR2 vaccine targeting the novel epitope created by pre-mRNA processing of the TNFR2 in which exon 7 is excluded joining exon 6 to exon 8 which provides a therapeutic option for those patients to which TNFα inhibitors are not recommended.

### SUMMARY

One embodiment described herein is a vaccine for immunizing a subject from an inflammatory condition or treating an inflammatory condition, wherein the vaccine comprises an mRNA vaccine encoding Δ7 or Δ7,8 splice variants of soluble TNF receptor II (sTNFR2) or a peptide vaccine comprising the amino acid sequence of Δ7 or Δ7,8 splice variants of sTNFR2. In one aspect, the mRNA vaccine comprises one or more nucleic acid sequences having at least 95-99% identity to the nucleotide sequences of SEQ ID NO: 1, 3, 5, 7, 9, 11, 13, 15, 17, or 19; and one or more pharmaceutically acceptable excipients. In another aspect, the mRNA vaccine comprises one or more nucleotide sequences of SEQ ID NO: 1, 3, 5, 7, 9, 11, 13, 15, 17, or 19. In another aspect, the nucleic acid sequence comprises one or more modifications to the ribose sugar, the nucleotide base, modifications at or near the 5′-terminus, modifications at or near the 3′-terminus, or combinations thereof. In another aspect, the modifications comprise phosphorothioate linkages, 2′-O-methyl ribonucleotides, 2′-O-ethyl ribonucleotides, 2′-methoxyethyl ribonucleotides, or 2′-F ribonucleotides, 5′-or 3′-termini modification with biotin, triethylene glycol (TEG), hexaethylene glycol (Sp18), 1,3-propanediol (SpC3), or replacement of the deoxy or ribose moiety with morpholino or 2′-oxygen-4′-carbon methylene. In one aspect, the peptide vaccine comprises one or more polypeptide sequences having at least 95-99% identity to the amino acid sequences of SEQ ID NO: 2, 4, 6, 8, or 10; and one or more pharmaceutically acceptable excipients. In another aspect, the peptide vaccine comprises one or more polypeptide sequences of SEQ ID NO: 2, 4, 6, 8, 10, 12, 14, 16, 18, or 20. In another aspect, the polypeptide sequence comprises one or more modifications to the peptide backbone, the amino acid chains, the N-terminus, the C-terminus, or combinations thereof. In one aspect, the vaccine is immediately released or controlled released.

Another embodiment described herein is a method for treating an inflammatory condition in a subject in need thereof or immunizing a subject from symptoms of an inflammatory condition by administering a therapeutically effective amount of the vaccine described herein to the subject. In one aspect, the subject is a mammal. In another aspect, the subject is a human, non-human primate, cow, horse, pig, sheep, goat, dog, cat, rabbit, rat, or mouse. In another aspect, the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis; arachnoiditis; neuritis; arthritis; dermatomyositis soft tissue: myositis; synovitis/tenosynovitis; bursitis; enthesitis; fasciitis; capsulitis; epicondylitis; tendinitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof. In another aspect, the subject has an inflammatory condition where TNFα inhibitors are contraindicated.

Another embodiment described herein is a method for administering a vaccine described herein, by contacting a subject's immunocytes with the vaccine. In one aspect, the administering is performed using a hypodermic needle, infusion, or a guided injection or infusion using ultrasound, CT, MRI, or fluoro imaging.

Another embodiment described herein is a kit comprising a vaccine described herein; a receptacle; optionally a means for administering the vaccine; optionally a label and/or instructions for use; and optionally tamper resistant packaging.

Another embodiment described herein is the use of the vaccine described herein for the preparation of a medicament for treating an inflammatory condition in a subject in need thereof or immunizing a subject from symptoms of an inflammatory condition. In another aspect, the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis; arachnoiditis; neuritis; arthritis; dermatomyositis soft tissue: myositis; synovitis/tenosynovitis; bursitis; enthesitis; fasciitis; capsulitis; epicondylitis; tendinitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof. In another aspect, the medicament is administered by contacting a subject's immunocytes with the vaccine. In another aspect, the subject has an inflammatory condition where TNFα inhibitors are contraindicated.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows identical and homologous amino acid residues in the Δ7 sTNFR2 and Δ7,8 STNFR2 polypeptides, respectively, at the Exon 6-Exon 8 junction in mice (SEQ ID NO: 2, 4), rats (SEQ ID NO: 6, 8) cats (SEQ ID NO: 10, 12), dogs (SEQ ID NO: 14, 16), and humans (SEQ ID NO: 18, 20). See Table 1.

Micrographs of the tissue sections for Group A (colitis only) are shown in FIG. 2A-D; Group B (adjuvant only) in FIG. 3A-C, and Group C (peptide vaccination) in FIG. 4A-C.

FIG. 2A-D show micrographs of rat colon tissue sections of Group A, the colitis model, stained with H&E (FIG. 2A, FIG. 2C) or Masson's trichrome (FIG. 2B, FIG. 2D). All micrographs are at 200x. The micrographs show extensive loss of the colon epithelium, destruction and absence of crypts, severe inflammatory cell infiltration, hemorrhages, and edema of the submucosa.

FIG. 3A-C show micrographs of rat colon tissue sections of Group B, colitis model with adjuvant only administered 10 days prior to initiating colitis. The sections are stained with H&E (FIG. 3A-B) or Masson's trichrome (FIG. 3C). All micrographs are at 200x.

FIG. 4A-C show micrographs of rat colon tissue sections of Group C, colitis model with the sTNR2 peptide, SEQ ID NO: 18 and adjuvant administered 10 days prior to initiating colitis.

The sections are stained with H&E (FIG. 4A-B) or Masson's trichrome (FIG. 3C). All micrographs are at 200x. The sTNR2 peptide provided significant protection against inflammation and the tissue appears healthy and normal.

FIG. 5A-B show photomicrographs of rat colon tissue sections of Group A (FIG. 5A) or Group C (FIG. 5B) 8 days after initiating colitis. The sections are stained with H&E, Masson's trichrome, and periodic acid-Schiff (PAS) stain. Immunohistochemistry was performed using a FoxP3 rabbit monoclonal antibody and a goat anti-rabbit secondary antibody conjugated with peroxidase.

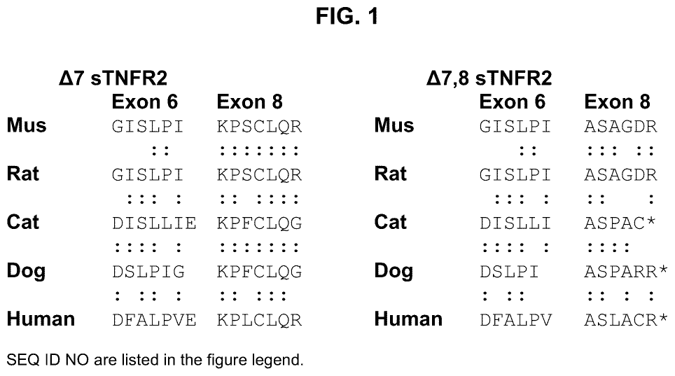

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified. As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refer to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. As used herein, a subject is “in need of treatment” if such subject would benefit biologically, medically, or in quality of life from such treatment. A subject in need of treatment does not necessarily present symptoms, particularly in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “antigen” is a molecule that will trigger an immune response, abbreviated by “Ag.” An Ag may originate from within the body (a self-protein) or from an external site (non-self). The immune system may not react to self-proteins due to negative selection of T-cells in the thymus during development.

As used herein, “negative selection” is a process in which lymphocytes, capable of strong binding with self-protein defined by the major histocompatibility complex (MHC) are removed by receiving an apoptosis signal leading to cell death. Some lymphocytes are phagocytosed by dendritic cells which allows presentation of self-antigens to MHC class II, a requirement for CD4+ T-cell negative selection. Some of these T-cells responding to self-proteins become Treg (T-regulatory) cells. The process is a component of central tolerance which prevents formation of cells capable of inducing autoimmune diseases.

As used herein, an “antibody” is a “Y” shaped protein, immunoglobulin (Ig), with an antigen binding site and an Fc region. Antibodies from humans include several classes or isotypes; IgA, IgD, IgE, IgG, or IgM. The IgG is composed of four polypeptide chains; two heavy chains and two light chains connected by disulfide bonds. The light chains contain one variable domain (VL) and one constant domain (CL), and the heavy chains contain one variable domain (VH) and three to four constant domains (CH1, CH2, CH3). Structurally, an antibody has two antigen binding fragments (Fab) composed of VL, VH, CL, and CH1 and an Fc fragment forming the trunk of the Y.

As used herein, a “T-cell” is a type of white blood cells, a lymphocyte, that plays a central role in the Adaptive immune response. T-cells are differentiated from other lymphocytes by the presence of a T-cell receptor (TCR) on the cell surface. Multiple classes of T-cells are defined; CD8 killer T-cells, CD4 helper T-cells, and regulatory T-cells. Each class of T-cell performs a different function often involving release of cytokines. All T-cells originate from c-kit+Sca1+ hematopoietic stem cells (HSC) that reside in the bone marrow.

As used herein, an “epitope” is a structural feature of an antigen that is an antigenic determinant that matches an antibody recognition site.

As used herein, “immunization” is the provision of immunity by any means, active or passive.

As used herein, “active immunization” is the administration of agents for induction of immunity that is long-lasting or at times, life-long.

As used herein, “passive immunization” is the administration of exogenously produced immune substances (e.g., convalescent serum, adoptive transfer of T-cells, or monoclonal antibodies) that dissipates with the turnover of the administered substances.

As used herein, a “vaccine” is the conveyance of antigens to elicit immune responses that are generally protective. Multiple approaches to vaccine design are known including attenuated virus, inactive virus, protein subunit, DNA vaccines, vectored vaccines, and mRNA vaccines.

As used herein, “vaccination” is the physical administration of a vaccine.

As used herein, “vaccine adjuvant” is a substance that increases and/or modulates the immune response to a vaccine antigen. Adjuvants can be inorganic compounds (potassium alum, aluminum hydroxide, aluminum phosphate, calcium phosphate hydroxide), oils (paraffin oil, peanut oil, squalene), bacterial products (Mycobacterium boris toxoids, lipopolysaccharides), plant products (saponins), cytokines (IL-1, IL-2, IL-12), and stimulators of innate immune responses by binding Toll receptors (TLR ligand including CpG motifs).

As used herein, “RNA” is a ribonucleic acid and a polymeric molecule essential for coding, decoding, regulation, and expression of genes. Cellular organisms use messenger RNA (mRNA) to convert genetic information as guanine (G), uracil (U), adenine (A), and cytosine (C) as triplets into selection of amino acids in synthesis of specific proteins.

As used herein, an “mRNA vaccine” is a type of vaccine that uses a copy of a messenger RNA (mRNA) to express an antigen to produce an immune response as well as stimulate innate immune responses.

Described herein is a single epitope-based vaccine targeting the epitope created by pre-mRNA processing of the TNFR2 in which exon 7 is excluded joining exon 6 to exon 8 (Δ7 STNFR2); alternately exons 7 and 8 are excluded joining exon 6 to exon 9 (Δ7,8 sTNFR2). Targeting this disease associated neoantigen, sTNFR2, spares the membrane bound TNFR2 and will result in fewer allergenic, reactogenic, and autoimmune responses. In addition, the small antigen vaccine can be produced using highly reproducible methods, will be lower cost to produce, is water soluble, and may be stored as a lyophilized powder without the need for refrigeration.

The sTNFR2 vaccine described herein does not directly target TNFα leaving acute immune responses to vaccines and formation of germinal centers intact. Pasparakis, J. Exp. Med. 184:1397-1411 (1996). The vaccine may provide a therapeutic option for those patients to which TNFα inhibitors are not recommended.

The TNFR2 gene is composed of 10 exons with cassette exons 1, 3, 4, and 7 indicating exclusion of these exons will result in a mature transcript that remains in reading frame. The exon structure of the TNFR2 gene and pre-mRNA is identical between human, dog, cat, mouse, and rat (Table 1) so exclusion of exon 7 will lead to synthesis of a splice variant that is orthologous to the human sTNFR2 variant. These observations support the use of rodent animal models for efficacy studies and support the design of companion animal vaccines.

### Design, Synthesis, and Production

### Pharmaceutical Vaccine Compositions

The amino acids indicated contain a single epitope for immune response that will not overlap with any wild-type protein epitopes to ensure selectivity for the indicated TNFα variant.

TABLE 1
Antigen mRNA and Peptide Sequences

| | | Peptide Sequence | SEQ ID |
|---|---|---|---|
| Name | mRNA Sequence (5′→3′) | (N→C) | NO |
| Δ7 sTNFR2-Mouse | GGCAUCAGCCUGCCCAUC/AAGCC | GISLPI/KPSCLQR^{‡} | 1, 2* |
| | CAGCUGCCUGCAGAGG^{†} | | |
| Δ7,8 sTNFR2-Mouse | GGCAUCAGCCUGCCCAUC/GCCAG | GISLPI/ASAGDR | 3, 4 |
| | CGCCGGCGACAGG | | |
| Δ7 sTNFR2-Rat | GGCAUCAGCCUGCCCAUC/AAGCC | GISLPI/KPSCLQR | 5, 6 |
| | CAGCUGCCUGCAGAGG | | |
| Δ7,8 sTNFR2-Rat | GGCAUCAGCCUGCCCAUC/GCCAG | GISLPI/ASAGDR | 7, 8 |
| | CGCCGGCGACAGG | | |
| Δ7 sTNFR2-Cat | GACAUCUCUCUCCUCAUUG/AGAA | DISLLIE/KPFCLQG | 9, 10 |
| | GCCCUUCUGUCUACAAGGA | | |
| Δ7,8 sTNFR2-Cat | GACAUCUCUCUCCUCAUUG/CCUC | DISLLIA/SPAC* | 11, 12 |
| | ACCUGCCUGCUGA | | |
| Δ7 sTNFR2-Dog | GACAUCUCGCUUCCGAUCG/AGAA | DISLPIG/KPFCLQG | 13, 14 |
| | GCCCUUCUGCCUCCAGGGA | | |
| Δ7,8 sTNFR2-Dog | GACAUCUCGCUUCCGAUCG/CCUC | DISLPIA/SPARR* | 15, 16 |
| | ACCUGCCCGCCGAUAA | | |
| Δ7 sTNFR2-Human | GACUUCGCCCUGCCCGUGG/AGAA | DFALPVE/KPLCLQR | 17, 18 |
| | GCCCCUGUGCCUGCAGAGA | | |
| Δ7,8 sTNFR2-Human | GACUUCGCCCUGCCCGUG/GCCAG | DEALPV/ASLACR | 19, 20 |
| | UCUGGCCUGCAGA | | |

^{†}The mRNA sequences for SEQ ID NO: 1, 3, 5, 7 are predicted from mouse and rat codon-optimized reverse translation of the peptide sequences using EMBOSS Backtranseq.
^{‡}The ″/″ indicates an exon splice site, italics indicate a termination codon.
*The odd numbered sequences are for the mRNA sequences and the even numbered sequences are the polypeptides encoded by the mRNAs.

The wild type human sTNFR2 mRNA sequence is described in NCBI Reference Sequence: NM_001066.3 and the encoded sTNRF2 polypeptide is described in NCBI Reference Sequence: NP_001057.1 which are incorporated by reference herein for such teachings.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities or coding.

Further embodiments described herein include nucleic acid molecules comprising polynucleotides having nucleotide sequences at least about 50%, 55%, 60%, 65%, 70%, 75%, 80%, 85%, 90%, 91%, 92%, 93%, 94%, 95%, 96%, 97%, 98%, or 99% identical, and preferably at least about 90-99%, or 100% identical to (a) nucleotide sequences, or degenerate, homologous, or codon-optimized variants thereof, encoding polypeptides having the amino acid sequences in SEQ ID NOs: 1, 3, 5, 7, 9, 11, 13, 15, 17, or 19; (b) nucleotide sequences, or degenerate, homologous, or codon-optimized variants thereof, encoding polypeptides having the amino acid sequences in SEQ ID NOs: 2, 4, 6, 8, 10, 12, 14, 16, 18, or 20; and (c) nucleotide sequences capable of hybridizing to the complement of any of the nucleotide sequences in (a) or (b) above and capable of expressing functional polypeptides of amino acid sequences in SEQ ID NOs: 2, 4, 6, 8, 10, 12, 14, 16, 18, or 20.

By a polynucleotide having a nucleotide sequence, for example, at least 90-99% “identical” to a reference nucleotide sequence encoding a functional polypeptide having amino acid sequences shown in SEQ ID NOs: 2, 4, 6, 8, 10, 12, 14, 16, 18, or 20 is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding a functional polypeptide of amino acid sequences of SEQ ID NOs: 2, 4, 6, 8, 10, 12, 14, 16, 18, or 20.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′-or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence. As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986).

For example, due to the degeneracy of the genetic code, one having ordinary skill in the art will recognize that a large number of the nucleic acid molecules having a sequence at least about 50%, 55%, 60%, 65%, 70%, 75%, 80%, 85%, 90%, 91%, 92%, 93%, 94%, 95%, 96%, 97%, 98%, or 99% identical, and preferably at least about 90-99% or 100% identical to the nucleic acid sequence shown in SEQ ID NO: 1, 3, 5, 7, 9, 11, 13, 15, 17, or 19, or degenerate, homologous, or codon-optimized variants thereof, that will encode functional polypeptides of amino acid sequences of SEQ ID NOs: 2, 4, 6, 8, 10, 12, 14, 16, 18, or 20.

Another embodiment described herein are modified polynucleotides of SEQ ID NO: 1, 3, 5, 7, 9, 11, 13, 15, 17, or 19 that have increased in vivo or in vitro stability. The polynucleotides may be modified on the ribose sugar modified at the 2′-position such as 2′-O-methyl, 2′-O-ethyl, 2′-methoxyethyl, 2′-F, eplacement of the ribose sugar with a morpholino subunit, or modifications to the phosphodiester backbone such as replacement with phosphorothioate linkages. For example, in some embodiments the nucleotides at or near the 5′-terminus, 3′-terminus, or combinations thereof, may be modified. In some embodiments, the modifications include phosphorothioate linkages (PS), 5′-or 3′-modifications such as biotin, triethylene glycol (TEG), hexaethylene glycol (Sp18), 1,3-propanediol (SpC3), 2′-O-methoxyethyl (MOE) ribonucleotides, 2′-O-methyl ribonucleotides (2′-OMe), 2′-fluoro (2′-F) ribonucleotides, or locked nucleic acids (LNA). “Locked nucleic acid” or “LNA” refers to a modified ribonucleotide comprising a methylene bridge bond linking the 2′ oxygen to the 4′ carbon of the ribose pentose ring:

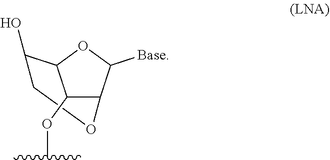

In one embodiment, one or more modifications are placed on the 5′-terminal nucleotide, the 5′-penulimate nucleotide, the 5′-antepenultimate (third) nucleotide, or a combination of the nucleotides at or near the 5′-terminus. In one embodiment, one or more modifications are placed on the 3′-terminal nucleotide, the 3′-penulimate nucleotide, the 3′-antepenultimate (third) nucleotide, or a combination of the nucleotides at or near the 3′-terminus. In another embodiment the modification is placed at the 2′-position of the 5′-terminal nucleotide, the 5′-penulimate nucleotide, the 5′-antepenultimate (third) nucleotide, or a combination of the nucleotides at or near the 5′-terminus. In another embodiment the modification is placed at the 2′-position of the 3′-terminal nucleotide, the 3′-penulimate nucleotide, the 3′-antepenultimate (third) nucleotide, or a combination of the nucleotides at or near the 3′-terminus. In additional embodiments the modification is placed at the 2′-position of the 5′-or 3′-terminal nucleotide, the 5′-or 3′-penulimate nucleotide, the 5′-or 3′-antepenultimate (third) nucleotide, or a combination of the nucleotides at or near the 5′-or 3′-termini. Such modifications may enhance stability or inhibit nuclease activity.

The polynucleotides described herein include those encoding mutations, variations, substitutions, additions, deletions, and particular examples of the polypeptides described herein. For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that proteins are surprisingly tolerant of amino acid substitutions.

The peptides may be modified to increase in vivo or in vitro stability. For example, the peptide termini may be modified such as N-alkylation, C-amidation, of D-amino acids, B-amino acids, or nonnatural amino acids can be incorporated, the peptide can be modified with carbohydrates (glycosylation), fatty-acids, or with synthetic moieties (e.g., PEGylation of termini or side chains), or by replacing typical peptide bonds with pseudo-peptide bonds (e.g., CO—NH replaced by NH—CO, CH₂—CH₂, or CO—CH₂).

Thus, fragments, derivatives, or analogs of the polypeptides of SEQ ID NO: 2, 4, 6, 8, 10, 12, 14, 16, 18, or 20 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 2, 4, 6, 8, 10, 12, 14, 16, 18, or 20 can be substituted with one or more conserved or non-conserved amino acid residue (preferably a conserved amino acid residue). In some cases, these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, or 99% identical, and preferably at least about 90-99% or 100% identical to the polypeptide sequence shown in SEQ ID NO: 2, 4, 6, 8, 10, 12, 14, 16, 18, or 20 and will comprise functional or non-functional proteins or enzymes. Similarly, additions or deletions to the polypeptides can be made either at the N-or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N-or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has at least 85% to 99% identity, including all integers within the specified range, to SEQ ID NO: 2, 4, 6, 8, 10, 12, 14, 16, 18, or 20. In another aspect, the polypeptide is selected from SEQ ID NO: 2, 4, 6, 8, 10, 12, 14, 16, 18, or 20.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by a method, or the means described herein. In one embodiment the nucleotides and polypeptides are produced synthetically by standard methods in the art.

Another embodiment described herein is the use of an effective amount of a polypeptide encoded by one or more of the nucleotide sequences described herein in SEQ ID NO: 2, 4, 6, 8, 10, 12, 14, 16, 18, or 20.

Another embodiment described herein is a research tool comprising a nucleotide sequence of SEQ ID NO: 1, 3, 5, 7, 9, 11, 13, 15, 17, or 19, or a polypeptide encoded by a nucleotide sequence of SEQ ID NO: 1, 3, 5, 7, 9, 11, 13, 15, 17, or 19, such as polypeptides of SEQ ID NO: 2, 4, 6, 8, 10, 12, 14, 16, 18, or 20.

Another embodiment described herein is a reagent comprising a nucleotide sequence of SEQ ID NO: 1, 3, 5, 7, 9, 11, 13, 15, 17, or 19, or a polypeptide encoded by a nucleotide sequence of SEQ ID NO: 1, 3, 5, 7, 9, 11, 13, 15, 17, or 19, such as polypeptides of SEQ ID NO: 2, 4, 6, 8, 10, 12, 14, 16, 18, or 20.

### Pharmaceutical Formulations

### Intravenous Injections

Phosphate buffered saline (PBS) containing inactive ingredients-potassium phosphate mono basic, anhydrous, USP; potassium chloride, USP; sodium phosphate diabetic, anhydrous, USP; sodium chloride, USP; and water for injection, USP. Active ingredients include mRNA with and without antisense IL-10E4SA. The phosphate buffered saline with and without enhanced delivery agents including lipid nanoparticles (LNP) including but not limited to lipids (including ((4-hydroxybutyl) azanediyl) bis (hexane-6, 1-diyl) bis (2-hexyldecanoate), 2 [(polyethylene glycol)-2000]-N,N-ditetradecylacetamide, 1,2-distearoyl-sn-glycero-3-phosphocholine, and cholesterol), perflourocarbon micro bubbles (C4F10 or C5F12), or cationic peptides including but not limited to ArgArgArgArgArgArgGly (R6G) (SEQ ID NO: 21).

### Intramuscular Injections

A PBS solution as described for intravenous injection including mRNA with and without antisense IL-10E4SA administered in a solution of less than one milliliter into a muscle, e.g., the shoulder. The PBS solution with or without enhanced delivery agents including lipid nanoparticles (LNP) or cationic peptides including but not limited to R6G (SEQ ID NO: 21).

### Inhalation

A PBS solution as described for intravenous injection including mRNA by an aerosol spray delivery device intra-nasally.

A dry powder composed of aerodynamic particle size distribution-a fine particle fraction (FPF) greater than 50%, a mean mass aerodynamic diameter (MMAD) of 2.0-2.5 micrometers, emitted dose (ED) of greater than 35% neat mRNA with and without IL-10E4SA and salt. The dry powder will be delivered by a flow-controlled inhalation metered device.

### Oral

An oral rehydration solution (ORT) composed of 2.6 g NaCl, 2.9 g trisodium citrate, 1.5 g potassium chloride, 13.5 g anhydrous glucose, and mRNA in one liter of water.

An oral solid dosage (pills or capsules) containing mRNA and excipients including binders, glidants, disintegrants, and lubricants to facilitate pill formation and dissolution in the gut.

### Formulations, Administration, Delivery, And Dosing Formulations

The linear mRNA can be formulated by multiple strategies indicated in this document. Patients seeking vaccination will have confirmed disease and will be screened for prior adverse reactions to vaccines to exclude patients that may experience serious adverse reactions. No concurrent chemotherapy is permitted due to likelihood of immune suppressive actions of chemotherapy that may limit the efficacy of a vaccine. Both male and female patients of all ethnic and ideological groups are included.

### Administration

The vaccine is administered by routes of administration described in this document such as intra-tumoral or intramuscular at doses of 0.03 to 0.10 mg of the mRNA or 0.1 to 0.5 mg/kg of peptide vaccine. The initial vaccination is designated day 0 and additional vaccinations will be administered at month 4, month 12, and month 24 to boost vaccine response. Patients are monitored for disease progression by standard procedures under the care of qualified medical personnel.

TABLE 2
Exemplary Administration Protocol

| Day | Day 0 | Month 4 | Month 12 | Month 24 |
|---|---|---|---|---|
| Dose | Initial | Booster+ | Booster+ | Booster+ |

### Delivery Methods

Direct pulmonary delivery (e.g., aerosol, inhalers, etc.) is a more selective mode of drug delivery that typically requires a lower quantity of drug. But it can have limited efficacy due to improper dosing, stability issues, and difficulty in producing an optimum particle size. Pulmonary drug delivery can provide the following advantages: quick onset of action coupled with ease and convenience of administration, the pulmonary dose is significantly lower than the oral dose, and degradation of the drug in the liver can be avoided. On the other hand, the following drawbacks are often associated with pulmonary drug delivery: improper dosing, stability problems, and difficulty in producing the optimum particle size. In addition, not all drugs can be delivered via a pulmonary route due to formulation difficulties.

Local therapeutic administration to solid tumors can be buttressed by using different types of drug delivery vehicles (nanoparticle drug carriers, liposomes, viral vectors, or microbubbles). The latter adhere to sites of damaged vascular endothelium and thus may be a method of systemically targeting delivery of therapeutics to damaged organs. For example, perfluorobutane gas microbubbles with a coating of dextrose and albumin efficiently bind to different pharmaceuticals. These 0.3-10.0 μm particles bind to sites of vascular injury. Further, the perfluorobutane gas is an effective cell membrane fluidizer. The potential advantages of microbubble carrier delivery include limited (additional) vessel injury through delivery, no resident polymer to degrade that may lead to eventual inflammation, rapid bolus delivery is feasible, and repeated delivery is possible. Microbubble carriers were successfully used in different animal models and clinical trials to deliver antisense oligonucleotide and/or Sirolimus to the injured vascular bed.

### Extracellular Vesicles (EV)

Kumar and colleagues describe the use of extracellular vesicles (EVs), which are a family of natural carriers in the human body. EVs play a critical role in cell-to-cell communications and can be used as unique drug carriers of therapeutic vaccine to tumors. However, the authors of the reported investigations concluded that certain limitations need to be overcome as well as requisite understanding of the mechanism to control targeted delivery. Specifically, the isolation and drug encapsulation techniques employed to engineer EVs could result in the loss of functional properties of the EVs, such as the destruction of surface proteins. These unintended changes could lead to nonspecific interactions with other cells, leading to off-target effects, toxicity, and suboptimal efficacy.

### Adenosine Nanoparticles

Recently, the efficacy in mitigating inflammation was demonstrated through the targeted delivery of adenosine and of multi-drug formulations. Bioconjugation of adenosine to squalene produces a prodrug-based nanocarrier, which, after nano formulation with Vitamin E, yields stable multidrug nanoparticles. This nanoparticle improves the bioavailability of both drugs with significant pharmaceutical activity in models of acute inflammatory injury.

### Novel Bio-Objects

A group of researchers has succeeded in engineering a new kind of microscopic bio-object that may one day be used for personalized diagnostics and targeted delivery of drugs. The object consists of a genetically modified E. coli bacterium and nano-erythrocytes (small vesicles made of red blood cells), and it demonstrates a substantial improvement in motility over previous designs.

### Nanobodies

In some embodiments therapeutic vaccine can be delivered using nanobodies. Indeed, several researchers have shown that nanobodies, which are tiny immune proteins, can enhance site specific delivery and residence of vaccines.

Nanomicelles

In brain tumors, vaccine may be delivered via brain-derived neurotrophic factor mRNA using polyplex nanomicelle.

Ischemic neuronal death causes serious lifelong neurological deficits; however, there is no proven effective treatment that can prevent neuronal death after the ischemia. We investigated the feasibility of mRNA therapeutics for preventing the neuronal death in a rat model of transient global ischemia (TGI). By intraventricular administration of mRNA encoding brain-derived neurotrophic factor (BDNF) using a polymer-based carrier, polyplex nanomicelle, the mRNA significantly increased the survival rate of hippocampal neurons after TGI, with a rapid rise of BDNF in the hippocampus.

The nanomicelle has a core-shell structure surrounded by a PEG outer shell and an mRNA-containing core for stable retention of the mRNA in the nanomicelle. The local administration of mRNA loaded nanomicelles has already shown therapeutic potential in various organs, such as the liver, joint cartilage, intervertebral disk, and the neural tissues, including the brain. Nanomicelles can also block the immune responses to extracellular mRNA by shielding them from recognition by the toll-like receptors in target cells.

In some embodiments the problems associated with systemic delivery may be overcome by using intra-arterial or intravenous selective delivery of therapeutic agents using a percutaneous trans-catheter route to deliver a therapeutic agent for treating solid organ tumors.

In some embodiments, a micro-catheter is introduced into a tumor feeding artery and/or its branches and a therapeutic agent for treating is infused through the micro-catheter. In some embodiments, the therapeutic agent is infused via an intravenous and/or intraarterial route using a micro-catheter.

In some embodiments, the therapeutic agent delivery via the micro-catheter is pressure-enabled using a continuous flow. In some embodiments, the therapeutic agent delivery via the micro-catheter is pressure-enabled using an automated pulsatile flow. Optionally, pumping the therapeutic agent out via the micro-catheter may be synchronized with the subject's breathing.

Different configurations of micro-catheters may be used with or without anti-reflux occluders including balloons, metallic constructions or fluid based vascular plugs. Any of the pharmaceuticals mentioned above may be delivered in a liquid state via the subject's blood, optionally with mixture of contrast agent and/or saline.

Alternatively, different carriers such as micro-particles, nanoparticles, injectable polymers or natural carriers and others may be used to enhance penetration and residence of therapeutics at the target area.

In some embodiments, a therapeutic vaccine is incorporated into substance-eluting beads (e.g., based on hydrogel and/or nanoparticles), and those beads are delivered via a micro-catheter using the blood vessels described above. The substance-eluting beads may be made, for example, by applying soybean lecithin to entrap hydrophilic bone morphogenic protein-2 into nanoporous poly (lactide-co-glycolide)-based microspheres. Other examples of materials that are suitable for forming substance-eluting beads include but are not limited to biocompatible, resorbable, or non-resorbable hydrogel beads. The beads may optionally be produced from polyvinyl alcohol and may contain a covalently bound radiopaque moiety. Saccharose beads, etc. may be used.

Other examples include silicon-based hydrogels; PEG-based polymers; nanoparticle-containing hydrogels; hydrogels containing cyclodextrins (CDs); hydrophilic polymers or poly ethylene glycol (PEG) provides water solubility to hydrogels, other polymers such as poly lactic acid (PLA), poly ϵ-caprolactone (PCL), polypropylene oxide (PPO), poly D,L-lactide-co-glycolide (PLGA) and poly ϵ-caprolactone-co-D,L-lactide (PCLA); ultra-thermosensitive hydrogel; hydrogels with different systems, namely, emulsions, vesicular (including micelles, liposomes and nanocapsules) and particulate systems (including mainly solid lipid micro and nanoparticles, nanostructured lipid carriers and lipid drug conjugates); biocompatible hydrogel, composed of the copolymer poly (N-isopropylacrylamide-co-n-butyl methacrylate) [P(NIPAAm-co-BMA)] and PEG; A polyethylenimine (PEI)-based hydrogel; supramolecular hydrogels; DNA-hydrogels; bioinspired hydrogels; and multi-functional and stimuli-responsive hydrogels; n-acrylic polymer impregnated with porcine gelatin; and gelatin.

In some embodiments, a therapeutic vaccine is incorporated into biological (Fibrin glue, fibrin matrix and collagen matrix) or different polymers drug delivery depots to increase residence of vaccine in target tumor area.

To avoid the disadvantages of oral or direct injection administration of drugs, a number of modes of administration of continuous dose, long-term delivery devices include reservoir devices, osmotic devices, and pulsatile devices for delivering beneficial agents have been utilized. Injecting drug delivery systems as small particles, microparticles or microcapsules avoids the incision needed to implant drug delivery systems. Microparticles, microspheres, and microcapsules, referred to herein collectively as “microparticles,” are solid or semi-solid particles having a diameter of less than one millimeter, more preferably less than 100 μm, which can be formed of a variety of materials, including synthetic polymers, and proteins. Another intensively studied polymeric injectable depot system is an in-situ-forming implant system. In situ-forming implant systems are made of biodegradable products, which can be injected via a syringe into the body, and once injected, congeal to form a solid biodegradable implant.

In some embodiment's the vaccine can be delivered by image guided percutaneous interventional procedures.

TABLE 3
Potential AtheroVax Uses

| Medical Area | Indication | References |
|---|---|---|
| Cardiovascular | heart failure, atherosclerosis myocarditis, | Schumacher, Curr. Cardiol. |
| | endocarditis, pericarditis, | Rep. 20(11): 117 (2018); |
| | cardiomyopathies, atrial fibrillation, | Iversen et al., Front Cardiovasc. |
| | valvular heart diseases, arteritis, | Med. 10: 1206541 (2023) |
| | phlebitis, capillaritis | |
| Oncology | breast, gastric, pancreatic, bladder, | Mercogliano et al., Cancers |
| | colorectal, oral, liver, leukemias, and | 13(3): 564 (2021). |
| | lymphomas | |
| | Combination with checkpoint inhibitors | |
| Rheumatology | rheumatoid arthritis, psoriatic arthritis, | Ciebiera et al., Int. J. Mol. |
| | ankylosing spondylitis myositis, bursitis, | Sci. 19(12): 3869 (2018) |
| | enthesitis, fasciitis, capsulitis, | |
| | epicondylitis, tendinitis, panniculitis, | |
| | periostitis | |
| Dermatology | plaque psoriasis | Ciebiera et al., Int. J. Mol. |
| | | Sci. 19(12): 3869 (2018) |
| Nephrology | glomerulonephritis, pyelonephritis, | |
| | diabetic nephropathy | |
| Pulmonology | asthma, adult respiratory distress, | Qin et al., Antisense Nucleic |
| | COPD, cigarette smoking sinusitis, | Acid Drug Dev. 10(1): 11-26 |
| | rhinitis, pharyngitis, laryngitis tracheitis, | (2000); Yao et al. Ther. Adv. |
| | bronchitis, bronchiolitis, pneumonitis, | Resp. Dis. 13: 1753466619866096 |
| | pleuritis | (2019); Zhao et al., PLoS |
| | | One 11(12): e0167451 (2016) |
| Ophthalmology | uveitis, dacryoadenitis, scleritis, | Ciebiera et al., Int. J. Mol. |
| | episcleritis, keratitis, retinitis, | Sci. 19(12): 3869 (2018) |
| | chorioretinitis, blepharitis, conjunctivitis | |
| Gastroenterology | Crohn's disease, ulcerative colitis, | Ciebiera et al., Int. J. Mol. |
| | esophagitis gastritis, gastroenteritis, | Sci. 19(12): 3869 (2018) |
| | enteritis, enterocolitis, duodenitis, ileitis, | |
| | cecitis, appendicitis, proctitis | |
| Gynecology | ovarian cancer, uterine fibroids | Ciebiera et al., Int. J. Mol. |
| | | Sci. 19(12): 3869 (2018) |
| Nervous System | Alzheimer's disease, spinal cord injury, | Ciebiera et al., Int. J. Mol. |
| | depression, encephalitis, myelitis, | Sci. 19(12): 3869 (2018); |
| | meningitis, arachnoiditis, neuritis | Feng, Ox. Med. Cell. Longev. |
| | | 2022: 8010670 (2022) |
| Musculoskeletal | arthritis, dermatomyositis soft tissue: | Ciebiera et al., Int. J. Mol. |
| system | myositis, synovitis/tenosynovitis, bursitis, | Sci. 19(12): 3869 (2018) |
| | enthesitis, fasciitis, capsulitis, | |
| | epicondylitis, tendinitis, panniculitis | |
| | osteochondritis: osteitis/osteomyelitis, | |
| | spondylitis, periostitis, chondritis | |
| Infectious | Hepatitis C Virus (HCV), human | Bader El Din, World J. |
| Diseases | papillomavirus (HPV), cytomegalovirus | Gastroenterol. 22(34): 7767- |
| | (CMV), SARS-CoV-2 (COVID-19) | 77 (2016); Hong et al., Int. J. |
| | | Oral. Sci. 12(1):3 (2020); |
| | | Imlay and Limaye, J. Infect. |
| | | Dis. 221(S1): S94-102 (2020); |
| | | Forte et al., 9(5): e01560-18 |
| | | (2018); Duan et al., Curr. Med. |
| | | Sci. 40(4): 618-624 (2020) |

### Post-Acute Sequelae of COVID-19 (Long COVID/PASC)

The health complications arising after recovery from COVID-19 are referred to as long COVID, post COVID-19 syndrome, or post-acute sequelae of COVID-19 (PASC). PASC is defined by the CDC as symptoms persisting for more than 28 days post the initial infection but other health organizations including the WHO require symptoms that persist for 12 weeks. The incidence of PSAC is estimated to be 10 to 43 percent of post COVID-19 cases. Multiple organ systems are involved. For example: (i) neurological sequelae include “brain fog,” anxiety disorder,

Guillain-Barre syndrome, impaired cognition, sense of smell, and fatigue loss of hearing; (ii) pulmonary functions associated with fibrotic remodeling and airspace obliteration such as respiratory insufficiency and failure; and (iii) with myocardial injury combined with coagulopathy, immunothrombus, cardiac arrhythmia, microvascular disfunction, vascular inflammation and heart failure.

The biological mechanisms are poorly understood but may involve: (i) SARS-COV-2 reservoirs in the body; (ii) presence of microthrombi; (iii) induction of autoantibodies, iv) hyperreactive immune activation; (v) mitochondrial dysfunction; and (vi) reactivation of latent viral infections such as Epstein-Barr Virus (EBV). However, the pathophysiology is associated with elevated C-reactive protein (CRP), Interleukin-6 (IL-6), and tumor necrosis factor-alpha (TNFα).

PASC represents a substantial public health concern. However, there are no current standards of care or consensus treatment options. Ongoing clinical trials designed to address PASC include evaluation of: (i) antiviral therapeutics such as favipiravir, nirmatrelvir, and remdesivir; (ii) cardiac agents such as ivabradine and metoprolol succinate; (iii) a wide variety of anti-inflammatory agents from cannabinoids to vitamin D; (iv) respiratory agents including montelukast, deupirfenidone, and bronchodilators; and (v) other classes of agents such as lithium, somatropin, and vortioxetine. Vaccination for SARS-COV-2 may improve PASC symptoms but in some cases, symptoms were worse. This list is not comprehensive but is included to demonstrate the spectrum of approaches.

TNFα is a key player in the pathophysiology of both acute COVID-19 and its post-acute sequelae. TNFα and IL-6 levels are elevated in individuals with PASC. Elevated levels of TNFα and IL-6 in individuals with PASC are reported across multiple studies.

Given the involvement of TNFα in both acute and post-acute phases of COVID-19, targeting this cytokine presents a potential therapeutic strategy. Anti-TNF therapies, such as infliximab and adalimumab, have been suggested as potential treatments to mitigate the inflammatory response in both acute COVID-19 and long COVID. Studies with AtheroVax targeting the sTNFR2 have demonstrated significant reductions in both TNFα and IL-6.

The role of tissue-resident TNF-alpha (TNFα) in PASC, produced by cells such as macrophages, dendritic cells, and fibroblasts, contributes to localized inflammatory responses and immune regulation within specific tissues. Tissue-resident TNFα may sustain chronic inflammation within specific organs affected by COVID-19, contributing to the persistent symptoms of long COVID. This can occur through continued activation of local immune cells, leading to ongoing tissue damage and dysfunction. Persistent tissue-resident TNFα can perpetuate a dysregulated immune response, potentially leading to autoimmunity. This can manifest as autoimmune-like symptoms, including joint pain, muscle aches, and rashes, which are often reported in long COVID. When sTNFR2 binds to TNFα, it forms a stable complex. This complex can serve as a reservoir for TNFα , sequestering it in a latent form. The TNFα-sTNFR2 complex can dissociate over time, releasing active TNFα back into the circulation or local tissues. This controlled release mechanism can contribute to sustained, low-level inflammatory signaling, potentially influencing chronic inflammation in long COVID. Enhancing the clearance of sTNFR2-TNFα complexes from the circulation might reduce the reservoir capacity, thereby limiting sustained TNFα signaling. Studies with AtheroVax targeting the sTNFR2 have demonstrated significant reductions in tissue resident TNFα and IL-6.

One embodiment described herein is a vaccine for immunizing a subject from an inflammatory condition or treating an inflammatory condition, wherein the vaccine comprises an mRNA vaccine encoding Δ7 or Δ7,8 splice variants of soluble TNF receptor II (sTNFR2) or a peptide vaccine comprising the amino acid sequence of Δ7 or Δ7,8 splice variants of sTNFR2. In one aspect, the mRNA vaccine comprises one or more nucleic acid sequences having at least 95-99% identity to the nucleotide sequences of SEQ ID NO: 1, 3, 5, 7, 9, 11, 13, 15, 17, or 19; and one or more pharmaceutically acceptable excipients. In another aspect, the mRNA vaccine comprises one or more nucleotide sequences of SEQ ID NO: 1, 3, 5, 7, 9, 11, 13, 15, 17, or 19. In another aspect, the nucleic acid sequence comprises one or more modifications to the ribose sugar, the nucleotide base, modifications at or near the 5′-terminus, modifications at or near the 3′-terminus, or combinations thereof. In another aspect, the modifications comprise phosphorothioate linkages, 2′-O-methyl ribonucleotides, 2′-O-ethyl ribonucleotides, 2′-methoxyethyl ribonucleotides, or 2′-F ribonucleotides, 5′-or 3′-termini modification with biotin, triethylene glycol (TEG), hexaethylene glycol (Sp18), 1,3-propanediol (SpC3), or replacement of the deoxy or ribose moiety with morpholino or 2′-oxygen-4′-carbon methylene. In one aspect, the peptide vaccine comprises one or more polypeptide sequences having at least 95-99% identity to the amino acid sequences of SEQ ID NO: 2, 4, 6, 8, or 10; and one or more pharmaceutically acceptable excipients. In another aspect, the peptide vaccine comprises one or more polypeptide sequences of SEQ ID NO: 2, 4, 6, 8, 10, 12, 14, 16, 18, or 20. In another aspect, the polypeptide sequence comprises one or more modifications to the peptide backbone, the amino acid chains, the N-terminus, the C-terminus, or combinations thereof. In one aspect, the vaccine is immediately released or controlled released.

Another embodiment described herein is a method for treating an inflammatory condition in a subject in need thereof or immunizing a subject from symptoms of an inflammatory condition by administering a therapeutically effective amount of the vaccine described herein to the subject. In one aspect, the subject is a mammal. In another aspect, the subject is a human, non-human primate, cow, horse, pig, sheep, goat, dog, cat, rabbit, rat, or mouse. In another aspect, the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis;

arachnoiditis; neuritis; arthritis; dermatomyositis soft tissue: myositis; synovitis/tenosynovitis; bursitis; enthesitis; fasciitis; capsulitis; epicondylitis; tendinitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof. In another aspect, the subject has an inflammatory condition where TNFα inhibitors are contraindicated.

Another embodiment described herein is a method for administering a vaccine described herein, by contacting a subject's immunocytes with the vaccine. In one aspect, the administering is performed using a hypodermic needle, infusion, or a guided injection or infusion using ultrasound, CT, MRI, or fluoro imaging.

Another embodiment described herein is a kit comprising a vaccine described herein; a receptacle; optionally a means for administering the vaccine; optionally a label and/or instructions for use; and optionally tamper resistant packaging.

Another embodiment described herein is the use of the vaccine described herein for the preparation of a medicament for treating an inflammatory condition in a subject in need thereof or immunizing a subject from symptoms of an inflammatory condition. In another aspect, the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis; arachnoiditis; neuritis; arthritis; dermatomyositis soft tissue: myositis; synovitis/tenosynovitis; bursitis; enthesitis; fasciitis; capsulitis; epicondylitis; tendinitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof. In another aspect, the medicament is administered by contacting a subject's immunocytes with the vaccine. In another aspect, the subject has an inflammatory condition where TNFα inhibitors are contraindicated.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A vaccine for immunizing a subject from an inflammatory condition or treating an inflammatory condition, wherein the vaccine comprises an mRNA vaccine encoding Δ7 or Δ7,8 splice variants of soluble TNF receptor II (sTNFR2) or a peptide vaccine comprising the amino acid sequence of Δ7 or Δ7,8 splice variants of sTNFR2.
Clause 2. The vaccine of clause 1, wherein the mRNA vaccine comprises one or more nucleic acid sequences having at least 95-99% identity to the nucleotide sequences of SEQ ID NO: 1, 3, 5, 7, 9, 11, 13, 15, 17, or 19; and one or more pharmaceutically acceptable excipients.
Clause 3. The vaccine of clause 1 or 2, wherein the mRNA vaccine comprises one or more nucleotide sequences of SEQ ID NO: 1, 3, 5, 7, 9, 11, 13, 15, 17, or 19.
Clause 4. The vaccine of any one of clauses 1-3, wherein the nucleic acid sequence comprises one or more modifications to the ribose sugar, the nucleotide base, modifications at or near the 5′-terminus, modifications at or near the 3′-terminus, or combinations thereof.
Clause 5. The vaccine of any one of clauses 1-4, wherein the modifications comprise phosphorothioate linkages, 2′-O-methyl ribonucleotides, 2′-O-ethyl ribonucleotides, 2′-methoxyethyl ribonucleotides, or 2′-F ribonucleotides, 5′-or 3′-termini modification with biotin, triethylene glycol (TEG), hexaethylene glycol (Sp18), 1,3-propanediol (SpC3), or replacement of the deoxy or ribose moiety with morpholino or 2′-oxygen-4′-carbon methylene.
Clause 6. The vaccine of clause 1, wherein the peptide vaccine comprises one or more polypeptide sequences having at least 95-99% identity to the amino acid sequences of SEQ ID NO: 2, 4, 6, 8, or 10; and one or more pharmaceutically acceptable excipients.
Clause 7. The vaccine of clause 1 or 6, wherein the peptide vaccine comprises one or more polypeptide sequences of SEQ ID NO: 2, 4, 6, 8, 10, 12, 14, 16, 18, or 20.
Clause 8. The vaccine of any one of clauses 1, 6-7, wherein the polypeptide sequence comprises one or more modifications to the peptide backbone, the amino acid chains, the N-terminus, the C-terminus, or combinations thereof.
Clause 9. The vaccine of any one of clauses 1, 6-8, wherein the vaccine is immediately released or controlled released.
Clause 10. A method for treating an inflammatory condition in a subject in need thereof or immunizing a subject from symptoms of an inflammatory condition by administering a therapeutically effective amount of the vaccine of any one of clauses 1-9 to the subject.
Clause 11. The method of clause 10, wherein the subject is a mammal.
Clause 12. The method of any one of clauses 10-11, wherein the subject is a human, non-human primate, cow, horse, pig, sheep, goat, dog, cat, rabbit, rat, or mouse.
Clause 13. The method of any one of clauses 10-12, wherein the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis; arachnoiditis; neuritis; arthritis; dermatomyositis soft tissue: myositis; synovitis/tenosynovitis; bursitis; enthesitis; fasciitis; capsulitis; tendinitis; epicondylitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof.
Clause 14. The method of any one of clauses 10-13, wherein the subject has an inflammatory condition where TNFα inhibitors are contraindicated.
Clause 15. A method for administering a vaccine of any one of clauses 1-9, by contacting a subject's immunocytes with the vaccine.
Clause 16. The method of clause 15, wherein the administering is performed using a hypodermic needle, infusion, or a guided injection or infusion using ultrasound, CT, MRI, or fluoro imaging.
Clause 17. A kit comprising a vaccine of any one of clauses 1-9; a receptacle; optionally a means for administering the vaccine; optionally an adjuvant; optionally a label and/or instructions for use; and optionally tamper resistant packaging.
Clause 18. Use of the vaccine of any one of clauses 1-9 for the preparation of a medicament for treating an inflammatory condition in a subject in need thereof or immunizing a subject from symptoms of an inflammatory condition.
Clause 19. The use of clause 18, wherein the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis; arachnoiditis; neuritis; arthritis; dermatomyositis soft tissue: myositis; synovitis/tenosynovitis; bursitis; enthesitis; fasciitis; capsulitis; epicondylitis; tendinitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof.
Clause 20. The use of clause 18 or 19, wherein the medicament is administered by contacting a subject's immunocytes with the vaccine.
Clause 21. The use of any one of clauses 18-20, wherein the subject has an inflammatory condition where TNFα inhibitors are contraindicated.

### EXAMPLES

### Example 1

### Δ7 sTNFR2 is a Favorable linear B-cell Epitope

The Δ7 sTNFR2 peptide must be a likely antigen in order for the vaccine to generate a favorable immune response. Predictive models for human B-cell epitopes, those likely to generate antibodies, have become reliable. We used the website, Tools.iedb.org/bcell/result to predict linear epitopes in the human Δ7 sTNFR2 peptide, DFALPVE/KPLCLQR (SEQ ID NO: 18; the “/” denotes the splice junction between exon 6 and exon 8). The simulation identified the VE/KPL peptide with a predictive score of 0.546 which exceeds the 0.500 epitope prediction threshold (Table 4). In this example, the linear epitope is exclusively within the splice junction between the protein encoded by the mRNA variant joining exon 6 to exon 8.

TABLE 4
Linear B-cell Epitope
Δ7 sTNFR2 peptide, DFALPVEKPLCLQR (SEQ ID NO: 18)
Predicted Peptides

| No. | Start | End | Peptide | Length |
|---|---|---|---|---|
| 1 | 6 | 10 | VEKPL | 5 |

Predicted Residue Scores

| Position | AA Residue | Score | Assignment |
|---|---|---|---|
| 0 | D | 0.244 | — |
| 1 | F | 0.302 | — |
| 2 | A | 0.362 | — |
| 3 | L | 0.428 | — |
| 4 | P | 0.489 | — |
| 5 | V | 0.520 | E |
| 6 | E | 0.533 | E |
| 7 | K | 0.544 | E |
| 8 | P | 0.546 | E |
| 9 | L | 0.540 | E |
| 10 | C | 0.483 | — |
| 11 | L | 0.422 | — |
| 12 | Q | 0.357 | — |
| 13 | R | 0.295 | — |

The assignment “E” indicates that the VEKPL residues are likely to be a B-cell epitope

### Example 2

### Efficacy in a Rat Colitis Model of Ulcerative Colitis

Rats were vaccinated with a human sTNFR2 peptide, DFALPVEKPLCLOR (SEQ ID NO: 18), targeting the human sTNFR2 peptide. The rats were injected with 25 μg of the peptide subcutaneously with 25 μg Alum as adjuvant in PBS 10 days before initiating colitis. The studies were performed in accordance with the recommendations of the Animal Care and Use Committee of Tbilisi State Medical University. Briefly, the colitis was induced by introduction of 4% acetic acid at 24-hour intervals for three days into the colon under light anesthesia (50 mg/kg ketamine HCl and 5 mg/kg xylaxine). Injections were intrarectal through 2 mm diameter, 6-8 cm long polyethylene catheter to deliver 1 mL solution. See Cagin et al., Exp. Ther. Med. 12 (5): 2958-2964 (2016).

The study design included 4 treatment groups:

Group A: colitis only

Group B: alum only 10 day prior, followed by colitis

Group C: sTNFR2 peptide (SEQ ID NO: 18)+alum 10 day prior, followed by colitis

Group D: no vaccine and no colitis

Visual observations indicate only rats in the sTNR2 peptide (SEQ ID NO: 18)+alum adjuvant significantly prevented loss of body weight, elevations in body temperature, and presented normal stools (Table 5).

TABLE 5
Rat Observations

| Animals | Stool | Bleeding |
|---|---|---|
| Group A (colitis only) | Diarrhea | Hemoccult Positive |
| Group B (adjuvant only) | Loose | Hemoccult Negative |
| Group C (vaccinated) | Normal | Hemoccult Negative |
| Group D (negative control) | Normal | Hemoccult Negative |

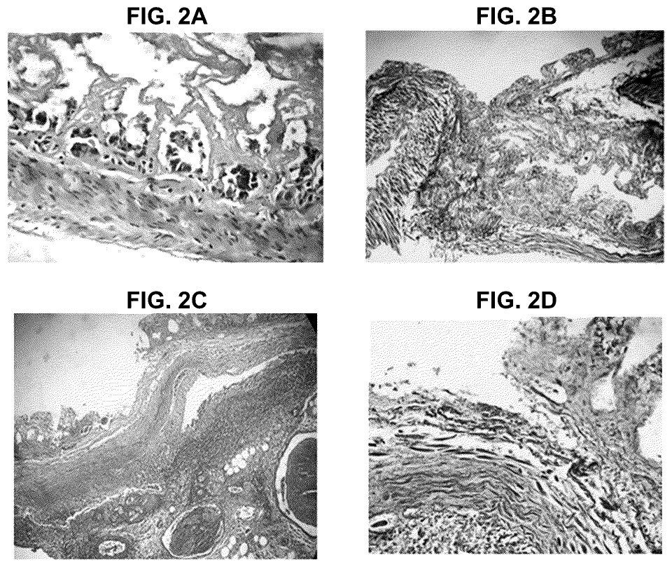

Examination of the colons from the rats indicate hyperemic, edematous, thickened and sharply increased volume in colons from rats in Group A (unvaccinated). See FIG. 2A-D. These gross anatomical observations were not associated with rats in Group C (sTNR2 peptide, SEQ ID NO: 18, +alum adjuvant). See FIG. 4A-C.

TNFα and IL-6 cytokines were measured with ELISA kits according to the manufacturer's instructions (Invitrogen). The ELISA plates were developed with tetramethylbenzidine (TMB) substrate and absorbance was read at 450 nm using an ELx808 Ultra Microplate Reader (BioTek Instruments GmbH, Lucerne, Switzerland). The measures of inflammatory markers identified significant reduction in TNFα in vaccinated animals (Table 6). Reduction in TNFα is consistent with loss of sTNFR2 binding.

All values reported are mean and standard error of the mean. Group comparisons are either two-tailed t-test or ANOVA calculated using Excel.

TABLE 6
Inflammatory Markers

| | TNFα | | | IL-6 | |
|---|---|---|---|---|---|
| Group | (mean ± SEM) | Comparison | p-values | (mean ± SEM) | p-values |
| A | 29.20 ± 0.65 | A vs. B | 1.12 × 10⁻⁶ *** | 6.46 ± 0.22 | 4.56 × 10⁻⁷ *** |
| B | 2.00 ± 0.05 | B vs. C | 9.29 × 10⁻⁴ *** | 2.54 ± 0.18 | 0.0050 ** |
| C | 1.12 ± 0.13 | A vs. C | 4.77 × 10⁻⁷ *** | 1.76 ± 0.13 | 4.38 × 10⁻⁷ *** |

** Significant difference, p-value ≤ 0.005 using a paired, two-tailed, T-test
*** Significant difference, p-value ≤ 0.001 using a paired, two-tailed, T-test

### Histology

Colon samples were fixed in 10% neutral buffered formalin for 24 hours at room temperature and embedded in paraffin. Tissue sections, 5 μm thick, were stained with hematoxylin and eosin (H&E), Masson's trichrome, and Periodic Acid-Schiff (PAS) according to the manufacturer's protocols. Histological evaluation of colons were assigned scores, (a score of 1 is normal and a score of 4 is severe pathology) for the number of animals with pathology >2, average pathology score, pathology distribution, and average inflammatory score (Table 7).

TABLE 7
Histology Scores

| | Number animals | Average | Pathology | Average |
|---|---|---|---|---|
| | with pathology >2 | Pathology | Distribution | Inflammatory |
| Group | (1-5) | Score (1-4) | (1-4) | Score (1-4) |
| A | 5 | 4 | 4 | 4 |
| B | 5 | 3 | 3 | 4 |
| C | 5 | 1 | 2 | 2 |

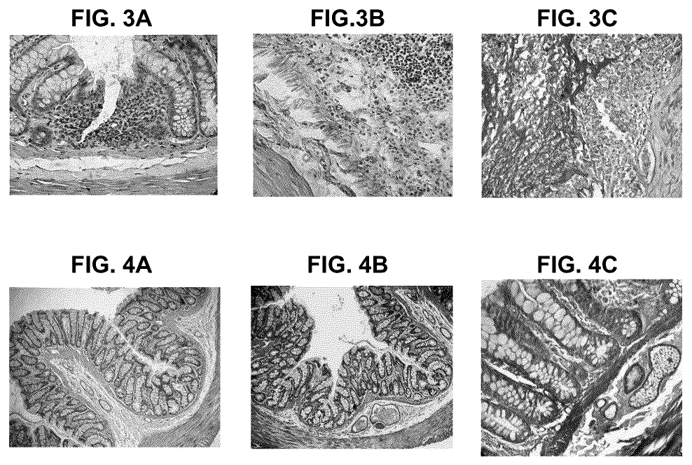

Micrographs of the tissue sections for Group A (colitis only) are shown in FIG. 2A-D; Group B (adjuvant only) in FIG. 3A-C, and Group C (peptide vaccination+adjuvant) in FIGS. 4A-C. The micrographs of the colitis model (Group A) and pre-treatment with the adjuvant only Group (B) showed significant loss of the colon epithelium, destruction of the crypts, severe inflammatory cell infiltration, hemorrhages, and edema of the submucosa. Vaccination with the sTNR2 peptide, (SEQ ID NO: 18) and alum adjuvant had a significant protective effect in preventing tissue loss and inflammation. See FIGS. 4A-C.

### Example 3

### Rat Ulcerative Colitis Model-Regulatory T Cells (Tregs)

These studies were performed in accordance with the recommendations of the Animal Care and Use Committee of Tbilisi State Medical University. Colitis was initiated following introduction of 4% acetic acid at 24-hour intervals for three days into colon under light anesthesia (50 mg/kg ketamine HCl and 5 mg/kg xylaxine). Injections were intrarectal through 2 mm diameter, 6-8 cm long polyethylene catheter to deliver 1 mL solution.

Colon samples were fixed in 10% neutral buffered formalin for 24 hours at room temperature and embedded in paraffin. Tissue sections 5 μm thick were stained with hematoxylin and eosin (H&E), Masson's trichrome, and Periodic Acid-Schiff stain (PAS) according to the manufacturer's protocols. The immunohistochemistry utilized a FoxP3 rabbit monoclonal antibody, ab215206, obtained from Abcam (Boston, MA) followed by a goat anti-rabbit secondary antibody conjugated with peroxidase.

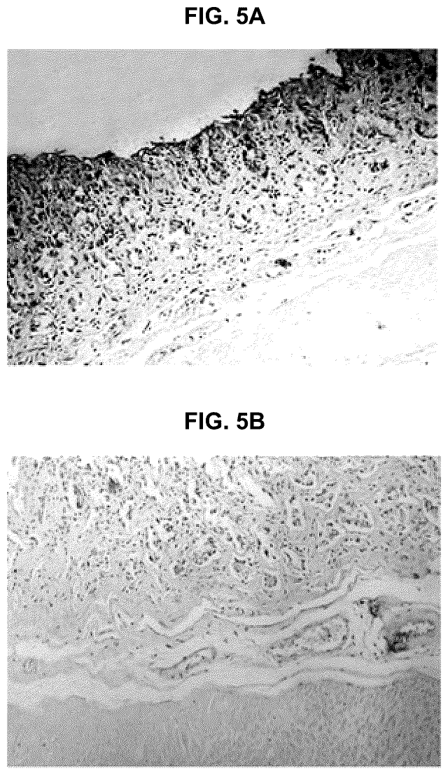

FIG. 5A shows a histological section of colon tissue from control/unvaccinated rats in which colitis was induced 8 days before collection of the colon tissue (Group A). The tissue is stained with antibodies (immunohistochemistry) that identify FoxP3 associated with identification of regulatory T cells (Tregs) that appear as intensely dark cells. The positively stained cells are localized in the epithelia bordering the lumen and the intestinal glands as well as the expected region of the lamina propria. FIG. 5B shows a histological section of colon tissue from rats vaccinated with 75 μg AtheroVax peptide (SEQ ID NO: 18) and the alum adjuvant one week prior to initiation of the colitis model (Group C). The colon tissue collected 8 days after initiation of colitis is stained with antibodies recognizing FoxP3 in an equal manner to tissues in FIG. 5A. The staining of Tregs is less intense and fewer cells are localized to the epithelia, the intestinal glands, and the lamina propria.

A segment of the colon was examined by immunohistochemistry staining for Tregs using an antibody recognizing FoxP3 eight days after initiation of the colitis model. Numerous cells in the unvaccinated control colons stain positive for FoxP3 primarily associated with the epithelia bordering the lumen, the intestinal glands, and extending into the lamina propria. The vaccinated colons do not stain as intensely for FoxP3 cells, and the number of positive cells is reduced compared to the vaccinated rats.

The anatomical extent of mucosal inflammation is one of the most important factors determining the course of ulcerative colitis. The frequency of CD4+FoxP3+ cells is higher in biopsy tissue from patients with extensive colitis and these cells may also express IL-17. The number of FoxP3+ cells is increased in inflammation involved ulcerative colitis tissue but have been found to be functionally impaired in counteracting inflammation.

Tregs are recruited to sites of chronic inflammation through the expression of specific chemokine receptors such as CCR4 and CCR6, which respond to chemokines produced in inflamed tissues. Once in the affected area, Tregs exert their regulatory functions to control the inflammatory response and promote tissue repair. The effectiveness of Tregs in chronic inflammatory environments depends on their ability to adapt to the local conditions and maintain their suppressive functions despite the pro-inflammatory signals.

TNFα and IL-6 promote Treg proliferation mediated by the TNFR2 pathway. TNFα and IL-6 promote T cell activation and increase resistance to Treg-mediated suppression. In humans, TNFα partially abrogated the suppressive function of CD4+CD25+ T cells isolated from chronic HBV-infected patients. Tregs from rheumatoid arthritis patients have been reported to lose FOXP3 expression and convert to pathogenic T cells when exposed to TNFα. TNFα-TNFR2 interaction downmodulated Treg suppressive function in an NF-KB activated pathway. TNFα blockade therapy paradoxically can exacerbate autoimmune pathologies indicating a regulatory role for TNFα in disease settings. IL-6 prevents differentiation of CD4+ T cells into peripherally induced Tregs. IL-6 can destabilize mouse thymic derived Tregs to produce IL-17. Human Tregs lose their immunosuppressive function following IL-6 exposure.

Observations from our studies in the rat model of ulcerative colitis find high frequency FoxP3 cells in the control colons. Significantly elevated levels of TNFα and IL-6 were observed both in circulating blood and in colon tissue. The control animals progressed to extensive damage to the colon tissue suggesting the enhanced Tregs had limited immunosuppressive function. Conversely, vaccinated animals were associated with significantly lower circulating and tissue resident levels of TNFα and IL-6 as well as lower frequencies of FoxP3 expressing cells in the colon. These observations are consistent with previous reports that high levels of TNFα and IL-6 are associated with proinflammatory Tregs and disease progression. The vaccine is associated with preserved integrity oof the colon and improved outcomes in the rat colitis model.

### Example 4

### Atherothrombosis Use

Adult Lewis rats weighing 200-250 g were randomly distributed into three treatment groups with 4 male and 4 female rats in each group. Group 1 received 1.0 mL saline subcutaneously on days 0 and 14. Group 2 received 1.0 mL saline with 0.025 mg AtheroVax peptide (SEQ ID NO: 18) and 0.025 mg aluminum hydroxide adjuvant on days 0 and 14. Group 3 received 1.0 mL saline with 0.075 mg AtheroVax peptide (SEQ ID NO: 18) and 0.075 mg aluminum hydroxide adjuvant on days 0 and 14. On day 21 the rats were anesthetized with ketamine and xylazine in the supine position, a laparotomy exposed the intestines and under a Zeis-Opton microscope the inferior vena cava was exposed and ligated 1 cm below the left renal vein using a 6/0 (Ethicon) ligature to create a thrombus. The abdominal cavity was sutured immediately after the ligation. The rats were euthanized 4 hours after dressing.

The thrombus was removed from the rats and wet weight determined. In addition, blood was recovered from the rats and coagulation parameters prothrombin time (PT), thrombin time (TT), and fibrinogen were measured.

Statistical analysis of mean and standard deviation for male, female, and combined values were calculated for each of the coagulation parameters. Pairwise comparisons using the students T-test were performed using Excel. Significance was determined if the p-value is less than 0.05.

No differences were detected between male and female rats in any of the parameters measured. The wet weight of the thrombus was significantly larger in Group 1 was 0.015±0.001 g, in Group 2 was 0.011±0.001 g, and in Group 3 was 0.007±0.001 g. Prothrombin time was significantly elevated in group 2 and 3 compared to group 1 (p<0.05), thrombin time was significantly decreased in groups 2 and 3 compared to group 1 (p<0.05) and fibrinogen was significantly increased in groups 2 and 3 compared to group 1 (p<0.05). See Table 8.

TABLE 8
Coagulation Parameters (combined male and female, N = 8/group)

| | | Grp. 2 | Grp. 3 | | | |
|---|---|---|---|---|---|---|
| | Grp. 1 | (25 μg | (75 μg | Grp 1 vs. | Grp 1 vs. | Grp 2 vs. |
| Parameter | (saline) | peptide) | peptide) | Grp. 2 | Grp. 3 | Grp. 3 |
| Prothrombin | 23.6 ± 0.80 | 24.5 ± 0.25 | 24.83 ± 0.15 | 8.27 × 10⁻³ | 5.79 × 10⁻⁴ | 3.39 × 10⁻³ |
| (sec) | | | | | | |
| Thrombin | 29.0 ± 0.42 | 26.2 ± 0.08 | 26.89 ± 0.20 | 1.99 × 10⁻¹¹ | 3.00 × 10⁻⁹ | 1.02 × 10⁻⁷ |
| (sec) | | | | | | |
| Fibrinogen | 27.8 ± 0.52 | 28.4 ± 0.15 | 29.3 ± 0.41 | 5.49 × 10⁻³ | 9.42 × 10⁻⁶ | 2.46 × 10⁻⁵ |
| (mg/L) | | | | | | |
| Thrombus | 0.015 ± 0.001 | 0.011 ± 0.001 | 0.007 ± 0.001 | 4.45 × 10⁻⁷ | 3.03 × 10⁻¹¹ | 2.63 × 10⁻⁶ |
| (grams) | | | | | | |

A thrombus (blood clot) is a healthy response to injury in which platelets and red blood cells aggregate in a mesh of cross-linked fibrin protein. However, a harmful thrombosis may form and obstruct blood flow in a healthy vessel. Uncontrolled platelet activation leading to thrombosis, atherothrombosis, is a main factor in unstable coronary syndromes and acute myocardial infarction.

Elevated levels of TNFα are associated with an increased risk of thrombotic events in various pathological conditions, including sepsis, rheumatoid arthritis, and atherosclerosis. The pro-thrombotic effects of TNFα highlight the interplay between inflammation and coagulation, contributing to our understanding of thrombus formation in inflammatory diseases. TNFα induces the expression of adhesion molecules such as E-selectin, ICAM-1, and VCAM-1 on endothelial cells, promoting endothelial cell activation and clotting. TNFα upregulates the expression of tissue factor (TF) on monocytes and endothelial cells. TF is a critical initiator of the extrinsic coagulation pathway, leading to thrombin generation and fibrin clot formation. TNFα a has been shown to activate platelets directly and indirectly. Activated platelets play a crucial role in thrombus formation by adhering to the site of vascular injury, aggregating, and providing a surface for the assembly of coagulation complexes. TNFα stimulates the release of procoagulant microparticles from various cells, including monocytes and endothelial cells. These microparticles can carry tissue factor and other procoagulant factors, contributing to thrombus formation. TNFα induces the production of other pro-inflammatory cytokines and chemokines, which can amplify the inflammatory response and promote a pro-thrombotic state. TNFα can impair fibrinolysis by increasing the expression of plasminogen activator inhibitor-1 (PAI-1), which inhibits tissue plasminogen activator (tPA) and urokinase, the enzymes responsible for fibrin degradation.

Treatment of rats with AtheroVax two weeks prior to initiation of thrombosis was associated with a dose dependent smaller thrombus weight, increased prothrombin times, decreased thrombin time, and elevation in fibrinogen.

1. A vaccine for immunizing a subject from an inflammatory condition or treating an inflammatory condition, wherein the vaccine comprises an mRNA vaccine encoding Δ7 or Δ7,8 splice variants of soluble TNF receptor II (sTNFR2) or a peptide vaccine comprising the amino acid sequence of Δ7 or Δ7,8 splice variants of sTNFR2.

2. The vaccine of claim 1, wherein the mRNA vaccine comprises one or more nucleic acid sequences having at least 95-99% identity to the nucleotide sequences of SEQ ID NO: 1, 3, 5, 7, 9, 11, 13, 15, 17, or 19; and one or more pharmaceutically acceptable excipients.

3. The vaccine of claim 2, wherein the mRNA vaccine comprises one or more nucleotide sequences of SEQ ID NO: 1, 3, 5, 7, 9, 11, 13, 15, 17, or 19.

4. The vaccine of claim 2, wherein the nucleic acid sequence comprises one or more modifications to the ribose sugar, the nucleotide base, modifications at or near the 5′-terminus, modifications at or near the 3′-terminus, or combinations thereof.

5. The vaccine of claim 4, wherein the modifications comprise phosphorothioate linkages, 2′-O-methyl ribonucleotides, 2′-O-ethyl ribonucleotides, 2′-methoxyethyl ribonucleotides, or 2′-F ribonucleotides, 5′-or 3′-termini modification with biotin, triethylene glycol (TEG), hexaethylene glycol (Sp18), 1,3-propanediol (SpC3), or replacement of the deoxy or ribose moiety with morpholino or 2′-oxygen-4′-carbon methylene.

6. The vaccine of claim 1, wherein the peptide vaccine comprises one or more polypeptide sequences having at least 95-99% identity to the amino acid sequences of SEQ ID NO: 2, 4, 6, 8, or 10; and one or more pharmaceutically acceptable excipients.

7. The vaccine of claim 6, wherein the peptide vaccine comprises one or more polypeptide sequences of SEQ ID NO: 2, 4, 6, 8, 10, 12, 14, 16, 18, or 20.

8. The vaccine of claim 6, wherein the polypeptide sequence comprises one or more modifications to the peptide backbone, the amino acid chains, the N-terminus, the C-terminus, or combinations thereof.

9. The vaccine of claim 1, wherein the vaccine is immediately released or controlled released.

10. A method for treating an inflammatory condition in a subject in need thereof or immunizing a subject from symptoms of an inflammatory condition by administering a therapeutically effective amount of the vaccine of claim 1 to the subject.

11. The method of claim 10, wherein the subject is a mammal.

12. The method of claim 10, wherein the subject is a human, non-human primate, cow, horse, pig, sheep, goat, dog, cat, rabbit, rat, or mouse.

13. The method of claim 10, wherein the inflammatory condition comprises one or more of: heart failure; atherosclerosis myocarditis; atherothrombosis; endocarditis; pericarditis; cardiomyopathies; atrial fibrillation; valvular heart diseases; arteritis; phlebitis; capillaritis; breast cancer; gastric cancer; pancreatic cancer; bladder cancer; colorectal cancer; oral cancer; liver cancer; leukemia; or lymphoma; rheumatoid arthritis; psoriatic arthritis; ankylosing spondylitis myositis; bursitis; enthesitis; fasciitis; capsulitis; epicondylitis; tendinitis; panniculitis; periostitis; plaque psoriasis; glomerulonephritis pyelonephritis; diabetic nephropathy; asthma; adult respiratory distress; COPD; cigarette smoking sinusitis; rhinitis; pharyngitis; laryngitis tracheitis; bronchitis; bronchiolitis; pneumonitis; pleuritis; uveitis; dacryoadenitis; scleritis; episcleritis; keratitis; retinitis; chorioretinitis; blepharitis; conjunctivitis; Crohn's disease; ulcerative colitis; esophagitis gastritis; gastroenteritis; enteritis; enterocolitis; duodenitis; ileitis; cecitis; appendicitis; proctitis; ovarian cancer; uterine fibroids; Alzheimer's disease; spinal cord injury; depression; encephalitis; myelitis; meningitis; arachnoiditis; neuritis; arthritis; dermatomyositis soft tissue: myositis; synovitis/tenosynovitis; bursitis; enthesitis; fasciitis; capsulitis; epicondylitis; tendinitis; panniculitis osteochondritis: osteitis/osteomyelitis; spondylitis; periostitis; chondritis; Hepatitis C Virus (HCV); human papillomavirus (HPV); cytomegalovirus (CMV); SARS-COV-2 (COVID-19), post-acute sequelae of COVID-19 (PASC), animal models of any of the foregoing conditions, or combinations thereof.

14. The method of 10, wherein the subject has an inflammatory condition where TNFα inhibitors are contraindicated.

15. A method for administering a vaccine of claim 1, by contacting a subject's immunocytes with the vaccine.

16. The method of claim 15, wherein the administering is performed using a hypodermic needle, infusion, or a guided injection or infusion using ultrasound, CT, MRI, or fluoro imaging.

17. A kit comprising the vaccine of claim 1; a receptacle; optionally a means for administering the vaccine; optionally an adjuvant; optionally a label and/or instructions for use; and optionally tamper resistant packaging.

18-21. (canceled)
